(19) United States
(12) Patent Application Publication
(10) Pub. No.: US 2017/0157393 A1
(43) Pub. Date: Jun. 8, 2017

(54) IMPLANTABLE INDIFFERENT ELECTRODE

(71) Applicant: Peter Osypka, Grenzach-Wyhlen (DE)

(72) Inventor: Peter Osypka, Grenzach-Wyhlen (DE)

(21) Appl. No.: 15/433,567

(22) Filed: Feb. 15, 2017

Related U.S. Application Data
(63) Continuation-in-part of application No. 14/359,839, filed on May 21, 2014, now abandoned.

(30) Foreign Application Priority Data
Dec. 3, 2011 (DE) 202011108639.2

(51) Int. Cl. A61N 1/05; A61N 1/362

(52) U.S. Cl. CPC A61N 1/0595; A61N 1/0563; A61N 1/3625

(57) ABSTRACT

An electrode arrangement, for the temporary stimulation or defibrillation of the heart includes at least one unipolar electrode configured to be secured on the myocardium of the heart. An indifferent electrode is configured to be secured on the inner wall of the thorax via its supply line and be positioned such that direct contact with the heart is prevented. An external pacemaker is in electronic communication with the at least one unipolar electrode and the indifferent electrode such that negative poles of the indifferent electrode are combined into a single cable.

### CROSS-REFERENCE TO RELATED APPLICATIONS

This application is a continuation-in-part of U.S. patent application Ser. No. 14/359,839, which is a U.S. National Stage Application under 35 U.S.C. §371 based on International Application No. PCT/DE2012/001108 filed Dec. 3, 2011, all of which are incorporated by reference herein in their entirety.

### BACKGROUND OF THE INVENTION

1. Field of the Invention

The present invention relates to an electrode arrangement, preferably for the temporary stimulation or defibrillation of the heart.

2. Description of Related Art

Catheters, tubes or electrodes are often left temporarily in place to facilitate the faster recovery after surgeries, especially after heart surgeries. In so doing, both the atriums as well as the chambers of the heart are stimulated electrically with an external pacemaker. Two unipolar electrodes (cardiac wires) or one bipolar electrode each are required for the stimulation of every heart chamber. If unipolar electrodes are used, the myocardium of the heart is injured in connection with the attachment of every pole. The outer adipose tissue of the heart often makes it difficult to locate a fat-free position required to attach the electrodes. Two insulated supply lines are always required for attaching bipolar electrodes on the heart. This is disadvantageous particularly for the hearts of children, because the weight or mass of the supply lines must be supported by the relatively small heart. Temporary electrodes attached on the heart after surgery are activated by a battery-operated external pacemaker. In the past, every electrode consisted of one indifferent and one different pole.

The cables often get tangled with the concurrent temporary stimulation of a plurality of heart chambers, thus compromising the reliability of the stimulation, particularly in connection with the dislocation of the poles. Mistakes frequently happen if the outward-leading lines are not clearly colour-coded for each heart chamber.

Even though four-chamber stimulation following heart surgery would be highly advantageous from an electrophysiological point of view, only what is urgently needed is often initiated for the patient because of the complexity associated with the different cables.

Therefore, the object is to provide an electrode arrangement, preferably for the temporary stimulation of the heart, which allows a reduction of the number of supply lines as compared to conventional arrangements.

To solve the object, the individual indifferent poles are combined to one large surface area pole positioned such that direct contact with the heart is prevented.

Depending on the wiring and design of the pacemaker, a shared indifferent electrode or a series of indifferent electrodes can be placed inside the body such that any direct contact with the heart is avoided or the impairment of its function is prevented.

The present invention therefore relates to an electrode arrangement, preferably for the temporary stimulation or defibrillation of the heart, consisting of at least one unipolar electrode, which is secured on the myocardium of the heart, and an indifferent electrode, which is secured on the inner wall of the thorax via its supply line and is positioned such that direct contact with the heart is prevented.

### SUMMARY OF THE INVENTION

Thanks to the arrangement of the combined indifferent electrode having a large surface area, only a single pole, i.e., only one supply line is required for the stimulation of the four heart chambers. This means that only five lines are required for the stimulation of the four heart chambers: one for the indifferent pole and four lines for each of the four different poles of the heart chambers. For the temporary application, the line to the indifferent pole can be inserted through the opening in the body both separately as well as together with the other poles.

Should the permanent implantation of a pacemaker be planned in the future, the indifferent electrode can also be designed as a permanent implant and connected temporarily with an external pacemaker via an adapter.

An electrode arrangement, for the temporary stimulation or defibrillation of the heart includes at least one unipolar electrode configured to be secured on the myocardium of the heart. An indifferent electrode is configured to be secured on the inner wall of the thorax via its supply line and be positioned such that direct contact with the heart is prevented. An external pacemaker is in electronic communication with the at least one unipolar electrode and the indifferent electrode such that negative poles of the indifferent electrode are combined into a single cable.

The indifferent electrode is metallic and preferably made of platinum, Nitinol, MP35N, a biocompatible stainless steel or conductive synthetic material.

As an example, the indifferent electrode can be a coil or provided as metal braiding, metal strand, metal wire, metal ribbon or metal weave.

The indifferent electrode can be embedded in a protective tube made of synthetic material, preferably silicone, said tube having openings toward the outside such as to allow an electrical current to flow between the unipolar electrode and the indifferent electrode through the openings of the tube.

The indifferent electrode can either be provided as a polished metallic coil or the coil can be embedded in a tube made of synthetic material, preferably silicone, said tube having openings. The openings are located on the side facing the heart, thus establishing insulation toward the side of the chest.

The indifferent electrode can also consist of metal braiding, which optionally comprises a membrane facing the side of the heart, said membrane preferably being made of silicone and having openings.

The outer shape, arrangement and size of the metallic coil can be selected to match the patient's anatomy. It is important that the indifferent electrode is arranged on the inside of the thoracic wall opposite the heart chambers such that the delivered defibrillator shock is fully effective.

The indifferent electrode can have different shapes. For instance, it can be designed straight, spiral-shaped, disc-shaped, shamrock-shaped, latticed, ribbon-shaped or polygonal or it can have the shape of a tennis racquet.

The indifferent electrode can comprise a membrane made of soft synthetic material on the side facing away from the heart. Aside from silicone, suitable membrane materials include polyurethane, PTFE, polyamide, Pebax and other biocompatible materials, which are suitable as long-term implants. This establishes insulation toward the side of the chest.

The supply lines for the indifferent electrode and the supply line for the unipolar electrode are secured for example by way of synthetic parts, preferably moulded silicone parts, secured by means of ligatures. The fixation can also be achieved with surgical adhesive. The line to the indifferent pole can be guided through the opening in the body both separately as well as together with the supply line to the unipolar electrode.

A further advantage of this electrode arrangement is that the electrodes (preferably coils) can be arranged parallel and pulled out easily from the body when the indifferent electrode is removed through the temporary opening in the chest.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

Additional specifics, characteristics and advantages of the invention can be gathered from the descriptive part below, in which the invention is explained in more detail based on the drawings and exemplary embodiments.

FIG. 1 shows the field strength distribution of an exemplary arrangement for stimulation of the heart with an indifferent electrode outside the heart;

FIG. 2a shows the prior art temporary dual chamber electrical heart stimulation with an external pacemaker after heart surgery;

FIG. 2b shows electrical heart stimulation with an external pacemaker according to the present disclosure;

FIG. 3 shows an exemplary view of the feed-through of an indifferent electrode through the thoracic wall;

FIG. 4 shows an exemplary large surface area disc-shaped indifferent electrode;

FIG. 5 shows a further exemplary indifferent electrode having a large surface area made from metal braiding;

FIG. 6 shows yet another exemplary indifferent electrode having a large surface area;

FIG. 7a shows one more exemplary indifferent electrode having a large surface area in the shape of a tennis racquet illustrating the indifferent electrode made of metal coils; and

FIG. 7b shows the indifferent electrode of FIG. 7a illustrating the coils coated with a silicone tube.

### DETAILED DESCRIPTION OF THE PREFERRED EMBODIMENTS

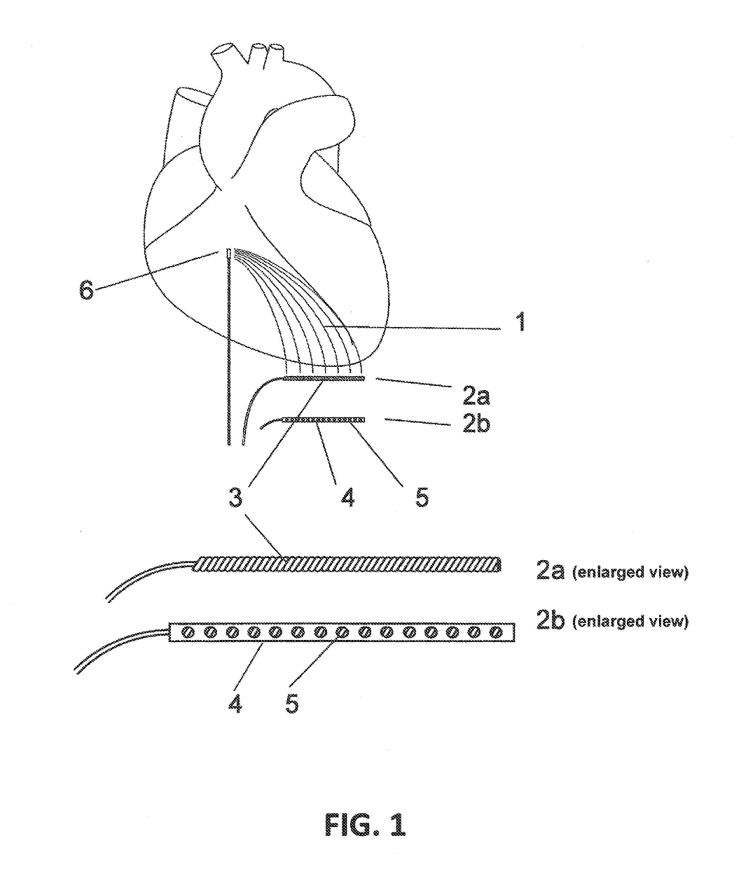

Reference will now be made to the drawings wherein like reference numerals identify similar structural features or aspects of the subject disclosure. For purposes of explanation and illustration, and not limitation, a partial view of an exemplary embodiment of an implantable indifferent electrode in accordance with the disclosure is shown in FIG. 1 and is designated generally by reference characters 2a and 2b.

FIG. 1 shows the field strength distribution 1 of an arrangement according to the present disclosure, wherein the indifferent electrode 2a, 2b is positioned outside the heart. The indifferent electrode 2a, 2b is created as metallic polished coil 3 in part 2a. However, the coil can also be embedded in a protective tube made of synthetic material, preferably silicone 4, said tube having openings 5 toward the outside such that an electrical current can flow through the openings between the unipolar electrode (cathode) 6 and the indifferent electrode 2a, 2b. The indifferent electrode 2a, 2b is positioned such that there is no direct contact of the electrodes during heart stimulation.

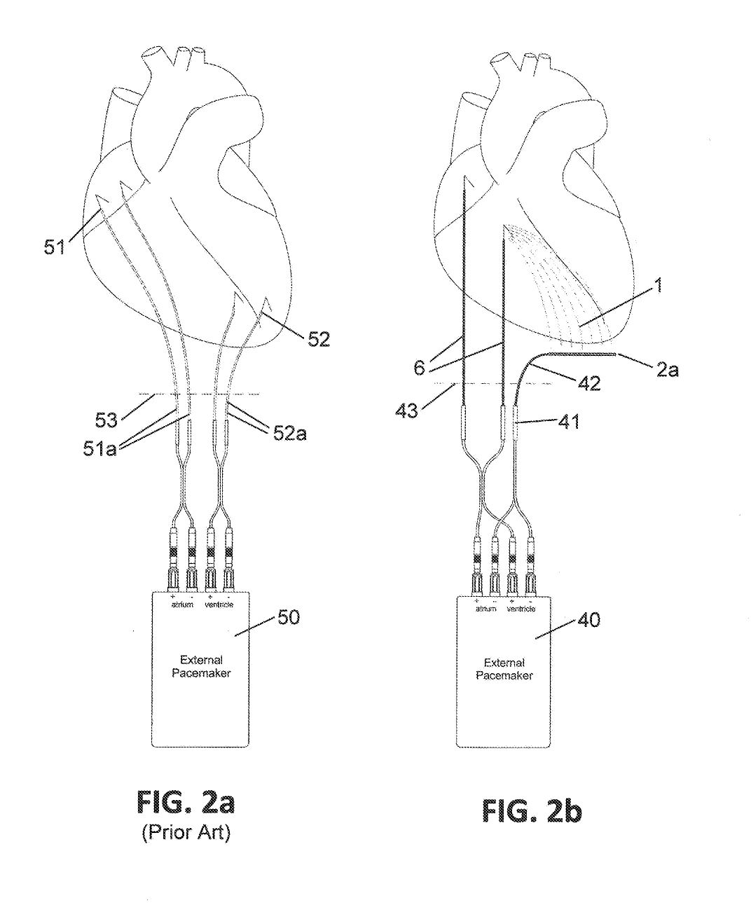

Referring to FIG. 2a a prior art temporary dual chamber electrical heart stimulation with an external pacemaker 50 after heart surgery is shown. The external pacemaker 50 stimulates alternately both chambers of the heart, the right atrium and the right ventricle, with two poles and therefore two unipolar wires for each chamber. Each pole 51, 52 of the wire has to be fixed within the wall of the heart (myocardium) in such a way as to allow the stimulation of the heart but also to allow the heart wires 51a, 52b to be removed through a percutaneous incision within the thorax region when their use is terminated.

In contrast, with reference to FIG. 2b, connection between the indifferent electrode 2a of the present invention and an external pacemaker 40 is shown. The external pacemaker 40 stimulates the right atrium and the right ventricle alternately at the end of an open heart surgical procedure to provide post-surgical temporary pacing and monitoring. The field strength distribution 1 between the right ventricle and the indifferent electrode 2a below the heart is shown during the stimulation of the right ventricle. A similar field strength appears when the right atrium is electrically stimulated. The end of the coil is an isolated lead cable 42 attached outside the abdominal wall 43 to a connector pin 41.

The present disclosure reduces the number of cables through the abdominal wall. In FIG. 2a (prior art) the unipolar electrode pole 51 fixed at the atrium and unipoloar electrode 52 fixed at the ventricle each need a counter electrode, thus four cables are hanging out from the abdomen 53. FIG. 2b shows that with the present disclosure the negative poles 41a of the indifferent electrode 2a are combined into one metal coil having one cable 42 through the abdomen wall 43. The metal coil is not fixed within the wall of the heart it is arranged near the apex of the heart.

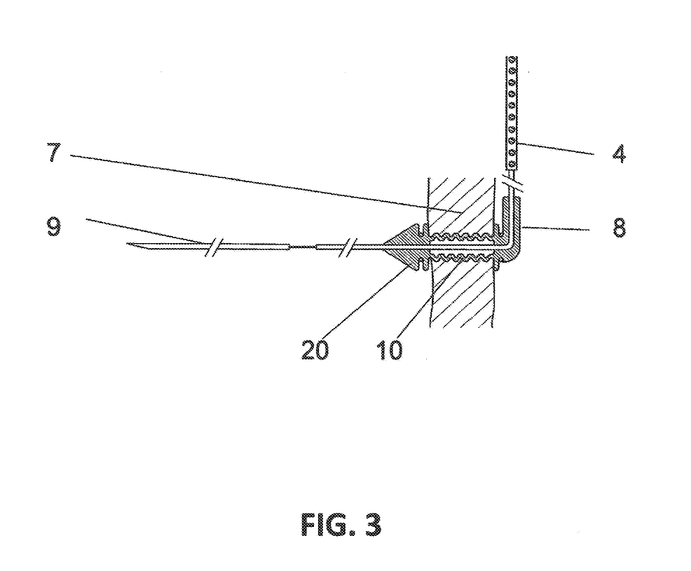

FIG. 3 shows an exemplary fixation of the indifferent electrode through the thoracic wall 7. The lead-through of the indifferent electrode through the thoracic wall 7 can be achieved with a moulded part made of silicone, wherein the moulded silicone part is fixated on the side of the electrode supply line by means of a ligature 20. A chest needle 9 is used to pull the indifferent electrode from the inside to the outside through the thoracic wall. Based on its undulating shape 10, the silicone part 8 can match the respective anatomical conditions of the chest wall thickness. The silicone part 8 can also be combined with the indifferent electrode 4 to form a joint moulded part.

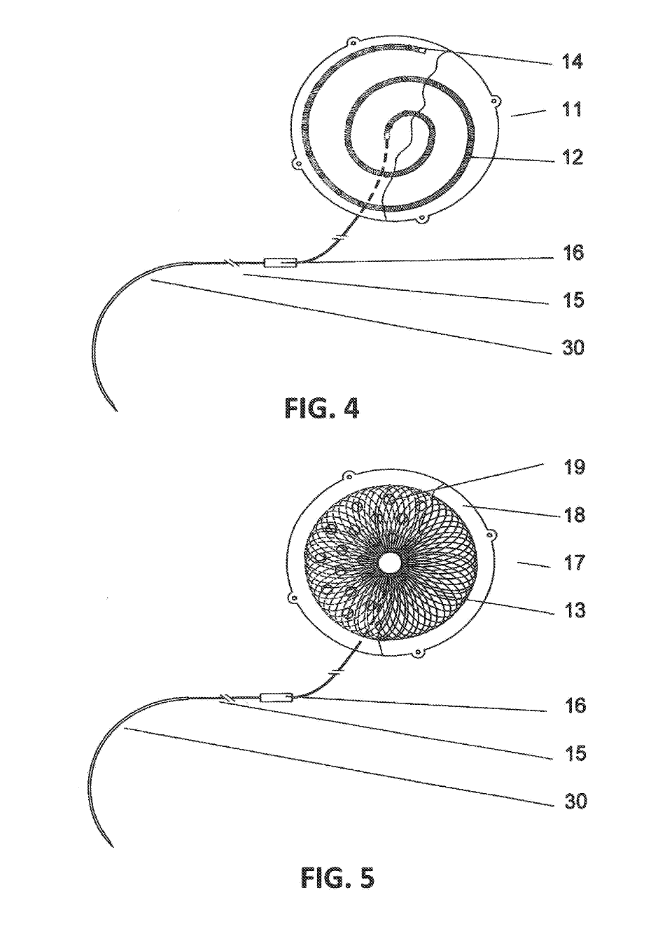

FIG. 4 shows an exemplary disc-shaped indifferent electrode having a large surface area 11. It serves as temporary indifferent electrode for the stimulation of the heart, but can likewise be used as permanent indifferent electrode for implanted pacemakers or implantable cardioverter-defibrillators (ICDs). To prevent direct contact between the coil (12) and the outer wall of the heart, the coil 12 can again, as described in FIG. 1, be integrated into a silicone tube having openings or into a silicone membrane having openings 14. The openings are necessary for the current to flow. A cable 15 connected with a detachable coupling 16, which is pushed through to the outside with a chest needle 30 is used to create the temporary connection with the external pacemaker.

FIG. 5 shows an additional exemplary indifferent electrode having a large surface area 17 made from metal braiding. It is a highly-flexible umbrella braided with individual Nitinol 13 wires. The umbrella is insulated toward the side of the chest with a silicone layer 18 and coated with a perforated silicone membrane 19 toward the side of the heart. A cable 15 connected with a detachable coupling 16 is again used to create the temporary connection with the external pacemaker.

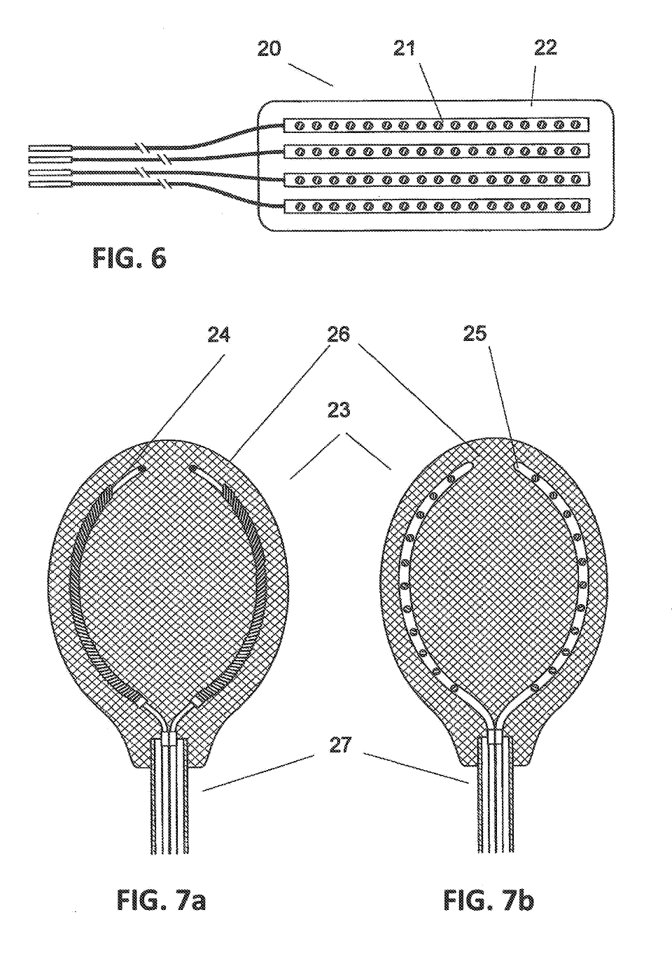

FIG. 6 shows a further exemplary indifferent electrode having a large surface area 20. The perforated silicone sections 21 are designed straight and can be connected as one or a plurality of indifferent electrodes as desired. Optionally, the silicone membrane 22 can be reinforced with fabric made of synthetic material (e.g. Dacron). The electrode is secured on the inside of the chest wall with ligatures, medical adhesive or other common surgical fixation devices. Metallic coils made of platinum, MP35N or other biocompatible conductive materials are used as poles. The supply line for the coil consists of a highly flexible internal metallic strand welded to the coil at the end. Metal braiding, metal weave, metal strand, metal ribbons or metal hemispheres or poles made of conductive synthetic material can be used aside from poles consisting of a coil. As well, the outer shape of the indifferent electrode can have a multiform design depending on the anatomical conditions (circular, rectangular, shamrock-like, latticed, in the shape of ribbons, squares, polygons, etc.). Aside from silicone, suitable membrane materials include polyurethane, PTFE, polyamide, Pebax and other biocompatible materials, which are suitable as long-term implants.

FIG. 7a and FIG. 7b show another exemplary indifferent electrode having a large surface area made of metal coils 24, arranged in the shape of a tennis racquet 23. The coils are insulated toward the side of the chest by a silicone membrane 26 and coated with a perforated silicone tube 25 toward the side of the heart. A cable 27 connected with a detachable coupling (not illustrated here) is again used to create the connection with the external pacemaker.

The system of the present disclosure, as described above and shown in the drawings, provide for an implantable indifferent electrode with superior properties including heart stimulation without the need for direct contact to the heart. While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. An electrode arrangement, for the temporary stimulation or defibrillation of the heart, comprising:
at least one unipolar electrode configured to be secured on the myocardium of the heart;
an indifferent electrode configured to be secured on the inner wall of the thorax via its supply line and be positioned such that direct contact with the heart is prevented; and
an external pacemaker in electronic communication with the at least one unipolar electrode and the indifferent electrode, wherein negative poles of the indifferent electrode are combined into a single cable.

2. An electrode arrangement according to claim 1, wherein the indifferent electrode is metallic and made of platinum, Nitinol, MP 35N, biocompatible stainless steel or conductive synthetic material.

3. An electrode arrangement according to claim 1, wherein the indifferent electrode is provided as a metal coil, metal braiding, metal strand, metal wire, metal ribbon or metal weave.

4. An electrode arrangement according to any one of claim 1, wherein the indifferent electrode consists of at least one metallic coil.

5. An electrode arrangement according to claim 4, wherein the metallic coil is embedded in a tube made of synthetic material, preferably silicone, said tube having openings, wherein the openings are located on the side facing the heart.

6. An electrode arrangement according to claim 1, wherein the indifferent electrode is made of metal braiding.

7. An electrode arrangement according to claim 6, wherein the metal braiding comprises a membrane having openings on the side facing the heart, said membrane preferably being made of silicone.

8. An electrode arrangement according to claim 1, wherein the indifferent electrode is designed straight, spiral-shaped, disc-shaped, shamrock-shaped, latticed, ribbon-shaped or polygonal or has the shape of a tennis racquet.

9. An electrode arrangement according to claim 1, wherein the indifferent electrode is secured in the patient's thoracic wall by means of a synthetic part.

10. An electrode arrangement according to claim 1, wherein the indifferent electrode comprises a membrane made of soft synthetic material, preferably silicone on the side facing away from the heart.

11. An electrode arrangement according to claim 1, wherein two positive poles from the unipolar electrode are positioned through the abdominal wall and in communication with the external pacemaker, and wherein one cable from the indifferent electrode is positioned through the abdominal wall and in communication with the external pacemaker.
